(19) United States
(12) Patent Application Publication
(10) Pub. No.: US 2007/0050030 A1
(43) Pub. Date: Mar. 1, 2007

(54) Expandable implant device with interchangeable spacer

(76) Inventor: Richard C. Kim, San Diego, CA (US)

Correspondence Address: Richard C. Kim, 13023 Polvera Avenue, San Diego, CA, 92128, US

(21) Appl. No.: 11/508,003

(22) Filed: Aug. 22, 2006

Related U.S. Application Data
(60) Provisional application No. 60/710,525, filed on Aug. 23, 2005.
(60) Provisional application No. 60/759,094, filed on Jan. 13, 2006.

(51) Int. Cl. A61F 2/44

(52) U.S. Cl. 623/17.11

(57) ABSTRACT

An implantable device that is expandable in vivo includes two end pieces and a spacer configured to be inserted between the two end pieces in vivo. The two end pieces are configured to be inserted simultaneously without the spacer. After the two end pieces have been inserted and positioned as desired, the spacer is inserted between the two end pieces in vivo to maintain a desired amount of separation between the two end pieces. The two end pieces each provide contoured surfaces for engaging and holding the spacer between the end pieces.

### RELATED APPLICATIONS

This application claims priority under 35 U.S.C. § 119(e) to U.S. provisional application Ser. No. 60/710,525, entitled “Expandable Cage With Interchangeable Spacer For Vertebral Replacement,” filed on Aug. 23, 2005, the entirety of which is incorporated by reference herein, and to U.S. provisional application Ser. No. 60/759,094, entitled “Magnetic Spinal Implants,” filed on Jan. 13, 2006, the entirety of which is also incorporated by reference herein.

### SUMMARY OF THE INVENTION

The present invention relates to spinal implant devices and methods of surgically implanting the devices. More particularly, the invention relates to a modular, expandable and implantable device for replacing one or more vertebral bodies and/or intervertebral disks, the device having a center piece (a.k.a., spacer) configured to be inserted between two end pieces in vivo so as to expand the two end pieces after they have been positioned between two vertebral bodies, thereby providing a desired level of distraction between the two vertebral bodies. The center piece is provided in various sizes and is interchangeable so that a single center piece can be inserted between the two end pieces to achieve a desired level of distraction between two vertebral bodies or other skeletal structures.

In one embodiment, the invention provides a method of inserting an implantable device having two end pieces and a spacer configured to be positioned between the two end pieces. The method includes simultaneously inserting the two end pieces into a surgical site of a patient; applying a force to separate the end pieces away from each other in vivo; inserting the spacer between the two end pieces while maintaining the separation force between the two end pieces; and releasing the separation force, wherein the spacer maintains a desired separation distance between the two end pieces.

In another embodiment, the invention provides an implantable device that is expandable in vivo and includes: a first end piece having a top surface and a contoured bottom surface; a second end piece having a bottom surface and a contoured top surface, wherein the first and second end pieces are configured to be inserted simultaneously into a surgical site of a patient with the contoured bottom surface of the first end piece facing the contoured top surface of the second end piece; and a spacer configured to be inserted between the first and second end pieces in vivo, the spacer having a first contoured surface configured to engage the contoured bottom surface of the first end piece, and a second contoured surface configured to engage the contoured top surface of the second end piece, wherein the spacer further includes a relatively narrow leading edge portion to facilitate insertion of the spacer between the first and second end pieces in vivo.

In a further embodiment, a method of providing an expandable spinal implant device that is customizable in real-time during surgery, includes: providing a first end piece having a top surface and a contoured bottom surface; providing a second end piece having a bottom surface and a contoured top surface, wherein the first and second end pieces are configured to be inserted simultaneously into a surgical site of a patient with the contoured bottom surface of the first end piece facing the contoured top surface of the second end piece; providing a plurality of interchangeable spacers of various heights, wherein each spacer is configured to be inserted between the first and second end pieces in vivo after the first and second end pieces have been inserted, each spacer having a first surface, configured to engage the contoured bottom surface of the first end piece, and a second surface configured to engage the contoured top surface of the second end piece, wherein each one of the plurality of spacers is configured to maintain a specified amount of separation between the first and second end pieces.

### BRIEF DESCRIPTION OF THE DRAWINGS

FIG. 1A illustrates a cross-sectional side view of a three-piece expandable implant device in accordance with one embodiment of the present invention.

FIG. 1B illustrates a perspective view of the expandable implant device of FIG. 1A when viewed from a perspective indicated by directional lines A-A of FIG. 1A.

FIG. 2A illustrates a perspective view of an interchangeable spacer, in accordance with one embodiment of the invention.

FIG. 2B illustrates a side view of the spacer of FIG. 2A.

FIG. 2C illustrates a cross-sectional side view of the end pieces, in accordance with one embodiment of the invention.

FIG. 3 illustrates another exemplary interchangeable spacer, in accordance with another embodiment of the invention.

FIG. 4 illustrates another exemplary spacer which is spherical or ellipsoidal in shape, in accordance with another embodiment of the invention.

FIG. 5 provides a perspective view of an exemplary end piece, in accordance with one embodiment of the invention.

FIG. 6A illustrates another exemplary end piece having a donut type shape, in accordance with one embodiment of the invention.

FIGS. 6B-6D illustrate exemplary views of a spacer configured to be placed between two end pieces, in accordance with one embodiment of the invention.

FIG. 7 illustrates a cross sectional view of an expandable implant device, in accordance with a further embodiment of the invention.

FIG. 8 illustrates a perspective view of the end piece of FIG. 7.

FIG. 9 illustrates a first insertion tool for inserting end pieces between two vertebral bodies, and a second insertion tool used for inserting a spacer in between the two end pieces, in accordance with one embodiment of the invention.

FIG. 10 illustrates a cross-sectional side view of the first end piece configured to receive a prong of the insertion tool of FIG. 9, in accordance with one embodiment of the invention.

FIG. 11A illustrates an end piece insertion tool that further includes a distance and/or force gauge, in accordance with one embodiment of the invention.

FIGS. 11B and 11C illustrate alternative embodiments of end prongs of the insertion tool of FIG. 11A.

FIGS. 12A-12C illustrate cross-sectional side views of an expandable implant device in accordance with additional embodiments of the invention.

FIGS. 13A-13C illustrate cross-sectional side views of an expandable implant device in accordance with further embodiments of the invention.

FIGS. 14A and 14B illustrate an expandable implant device including a top plate, in accordance with a further embodiment of the invention.

FIGS. 15A and 15B illustrate an expandable implant device including a top plate, in accordance with another embodiment of the invention.

FIG. 16 illustrates a cross-sectional side view of an expandable implant configured for intervertebral disk replacement, in accordance with one embodiment of the invention.

FIG. 17 illustrates a cross-sectional side view of an expandable implant configured for intra-vertebral augmentation, in accordance with one embodiment of the invention.

FIG. 18 illustrates a cross-sectional side view of an expandable implant having magnetic elements therein, in accordance with one embodiment of the invention.

FIG. 19 illustrates a block diagram of a power module that may be used to supply power to an electro-magnet contained with an expandable implant, in accordance with one embodiment of the invention.

FIGS. 20A and 20B illustrate cross-sectional side views of the end pieces and spacer of FIGS. 6A-6D having retractable gripping pins in the end pieces, in accordance with one embodiment of the invention.

### DETAILED DESCRIPTION OF THE PREFERRED EMBODIMENTS

The invention is described in detail below with reference to the figures, wherein like elements are referenced with like numerals throughout.

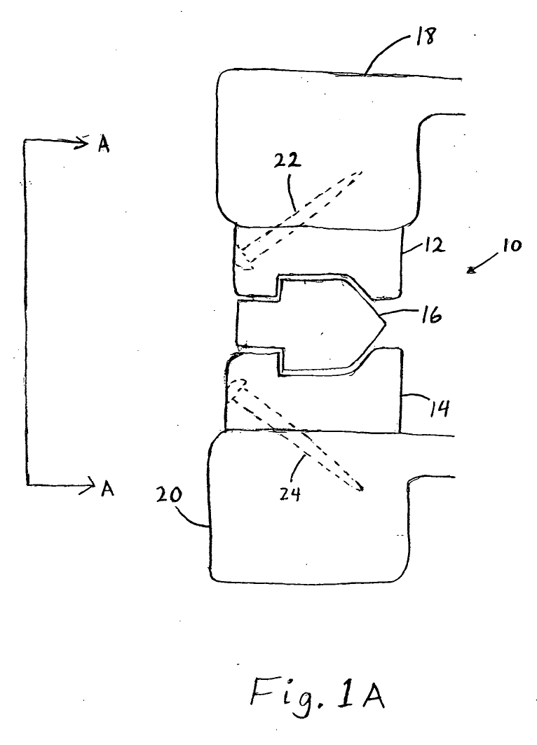

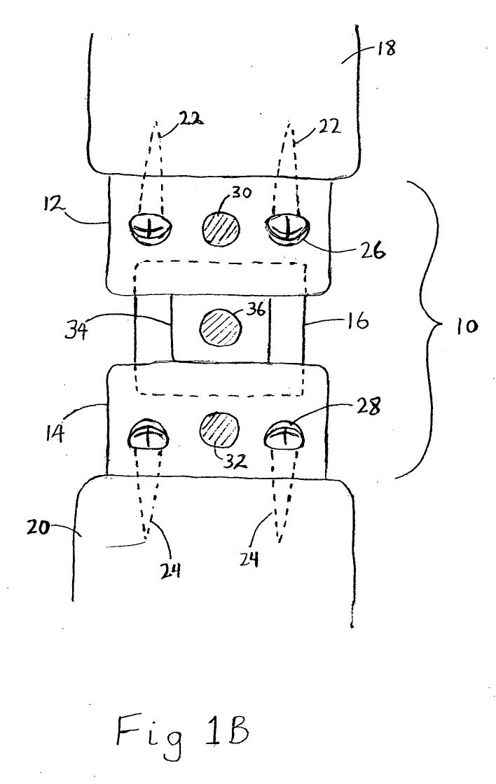

FIG. 1A illustrates a cross-sectional side view of a three-piece expandable implant 10 in accordance with one embodiment of the present invention. The expandable implant 10 includes a first end piece 12, a second end piece 14, and a spacer 16 configured to snugly fit within a shaped space between the first and second end pieces 12 and 14. The space or volume created between the first and second end pieces 12 and 14 has a geometric shape that is complementary to the shape of the spacer 16 such that once the spacer 16 is inserted between the end pieces 12 and 14, the spacer 16 is interlocked into position between the end pieces 12 and 14.

The first end piece 12 is configured to be positioned adjacent to a first vertebra 18 of a patient's spine and the second end piece 14 is configured to be positioned adjacent to a second vertebra 20 of the patient's spine. The vertebra 18 and 20 may be vertebral bodies in any area of the spine, such as the cervical, thoracic or lumbar areas of the spine. In one embodiment, the first end piece 12 is secured to the first vertebra 18 by at least one biocompatible surgical screw 22. Similarly, the second end piece 14 is secured to the second vertebra by at least one biocompatible surgical screw 24. As shown in FIG. 1, the surgical screws 22 and 24 are inserted through their respective end pieces 12 and 14 at a specified angle (e.g. 30 to 60 degrees from horizontal) and penetrate their respective vertebral bones 18 and 20 at the specified angle, thereby securing the end pieces 12 and 14 to the vertebras 18 and 20, respectively.

It should be understood that the Figures described herein are not necessarily drawn to scale and that the sizes and shapes of the various parts and elements illustrated in each Figure are only exemplary. Additionally, the relative sizes and shapes of some parts in the Figures may be exaggerated for purposes of illustration. Additionally, it is understood that the orientation of the expandable implant device 10 and the direction of insertion of the spacer 16 with respect to a patient's spine as illustrated in the Figures is also exemplary. In various embodiments of the invention, the orientation of the expandable implant device 10 and the direction of insertion of the spacer 16 can be configured in accordance with any desired surgical approach, such as straight anterior, anterolateral, lateral, posterolateral or posterior approaches.

FIG. 1B illustrates a perspective view of the expandable implant 10 of FIG. 1A when viewed from a perspective indicated by directional lines A-A of FIG. 1A. As shown in FIG. 1B, the first end piece 12 includes two angled screw holes 26 through which biocompatible screws 22 pass to penetrate vertebra 18 and secure the first end piece 12 to the vertebra 18. Similarly, the second end piece 14 includes two angled screw holes 28 through which biocompatible screws 24 pass to penetrate second vertebra 20 and secure the second end piece 14 to the second vertebra 20.

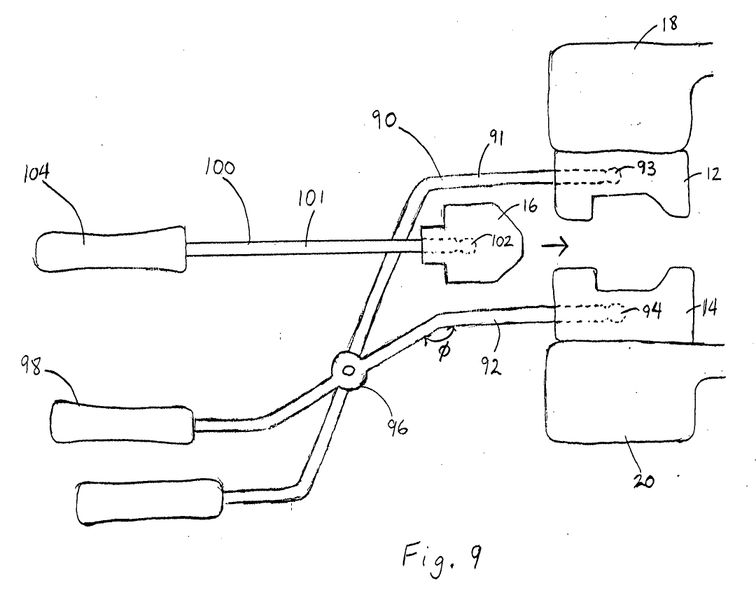

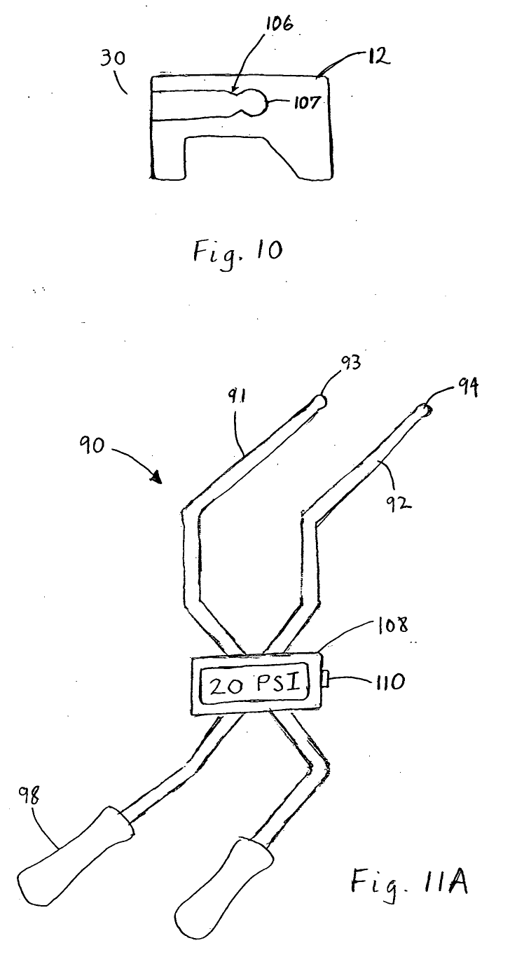

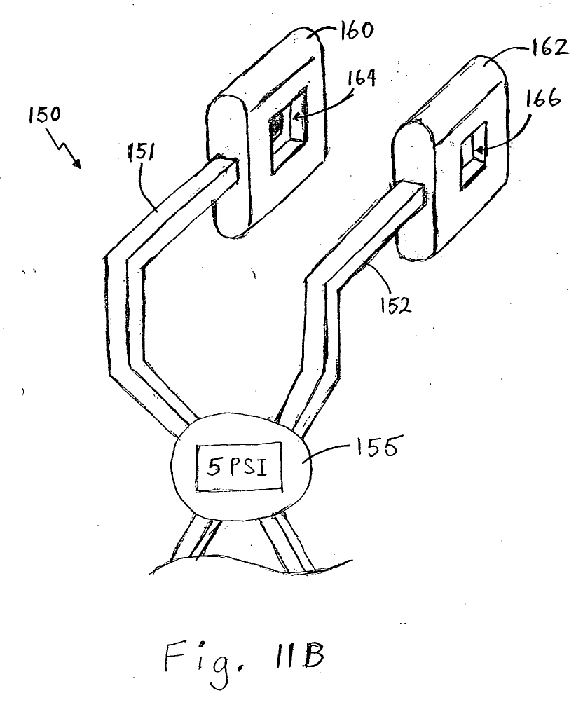

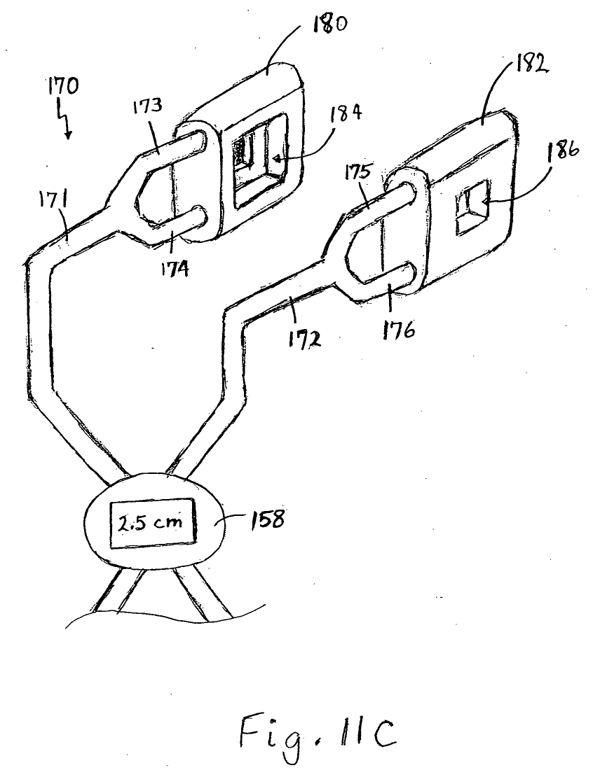

The first end piece 12 includes a first insertion hole 30 configured to receive a first prong of an end piece insertion tool 90 (FIGS. 9 and 10). The second end piece 14 also includes an insertion hole 32 configured to receive a second prong of the insertion tool. The functionality of the insertion tool and the holes 30 and 32 are described in further detail below with reference to FIGS. 9 and 10.

The interchangeable spacer 16 is securely locked into the shaped cavity of each end piece 12 and 14 as indicated by the dashed lines in FIG. 1B. The spacer 16 includes a rear portion 34 that is not located within the shaped cavity but can be seen located between the first and second end pieces 12 and 14, as shown in FIG. 1B. This rear portion 34 includes an insertion hole 36 configured to receive a prong of a spacer insertion tool 100 (FIG. 9) that is used to push the spacer 16 between the first and second end pieces 12 and 14. The functionality of the insertion hole 36 and the spacer insertion tool 100 is described in further detail below with reference to FIG. 9. Although the shape of the holes 30, 32 and 36 are illustrated as circular in FIGS. 9 and 10, these holes and the corresponding channels formed in their respective pieces 12, 14 and 16, may be any desired shape (e.g., square, oval, rectangular). In one embodiment, the shape of the holes/channels 30, 32 and 36 are square to provide improved rotational control of each end piece 12, 14 and/or spacer 16, by correspondingly shaped prongs 91, 92 and 101 of insertion tools 90 and 100 (FIG. 9).

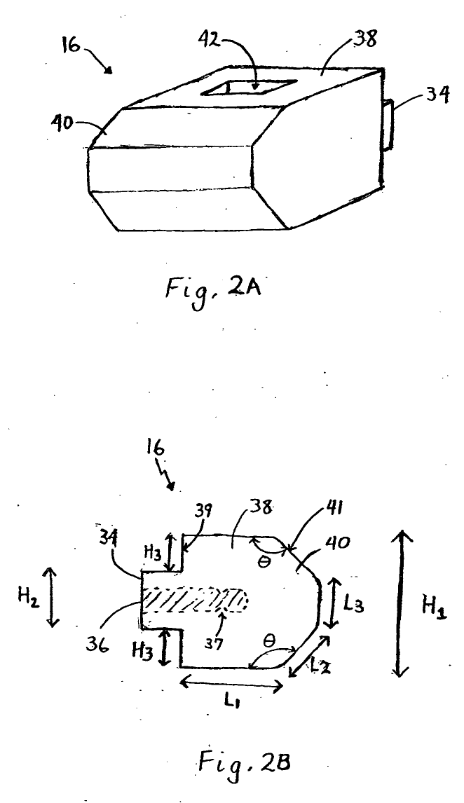

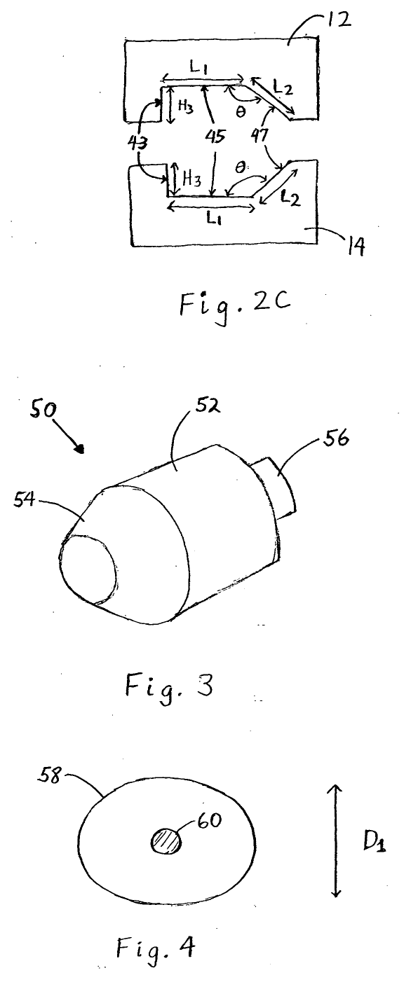

FIG. 2A illustrates a perspective view of an interchangeable spacer 16, in accordance with one embodiment of the invention. The spacer 16 includes a body portion 38, a gradually narrowing nose or front portion 40 extending from the body portion 38, and a rear portion 34 extending outwardly from the rear of the body portion 38. The narrow nose or front portion 40 facilitates insertion of the spacer 16 between the end portions 12 and 14 by gradually distracting the space between the end pieces 12 and 14 as the spacer 16 slides into the space. As shown in FIG. 2A, the rear portion 34 has a smaller vertical thickness or height than the body portion 38. In one embodiment, the spacer 16 includes a window 42 cut or formed through the body portion 38 to allow for insertion of bone growth material and/or otherwise promote bone growth through the spacer 16. Additional windows, cut-outs or holes (not shown) may also be incorporated into the spacer 16 for allowing and promoting bone growth through the spacer 16.

FIG. 2B illustrates a side view of the spacer 16 of FIG. 2A. The spacer 16 includes an insertion tool hole 36 that extends into the body of the spacer 16. The hole or channel 36 includes an indented portion 37 to provide a “snap-lock” mechanism that engages a corresponding “snap-lock” mechanism on a prong 101 of a spacer insertion tool 100, described in further detail below with respect to FIG. 9.

The body portion 38 of the spacer 16 has a height of H₁ and the rear portion 34 has a smaller height of H₂. Where the rear portion 34 meets the body portion 38 two opposing step walls 39 on opposite sides of the rear portion 34 each have a height of H₃. As shown in FIG. 2B, in one embodiment, the height of the body portion 38 (H₁) is equal to the height of the rear portion 34 (H₂) plus each of the heights of the step walls 39 (H₃). Thus, H₁=H₂+(2×H₃). Furthermore, the body portion 38 has a length of L₁ and angled walls 41 of the nose portion 40 has a length of L₂. The angled walls 41 are inclined at an angle from horizontal by an absolute value equal to θ degrees. If the spacer 16 possesses the exemplary shape and dimensions illustrated in FIG. 2B, in one embodiment, the first and second end pieces 12 and 14, respectively, includes corresponding shapes and dimensions in the cavity between the end pieces 12 and 14 where the spacer 16 is configured to fit.

FIG. 2C illustrates a cross-sectional side view of the end pieces 12 and 14, in accordance with one embodiment of the invention. Each of the end pieces 12 and 14 have a shaped cavity, each shaped cavity having an internal back-stop wall 43 with a height of H₃ corresponding to the height H₃ of the step wall 39 of the spacer 16. The shaped cavity further includes an internal longitudinal wall 45 having a length of L₁ and an internal inclined wall 47 having a length of L₂. The angle between the internal inclined wall 47 and the longitudinal wall 45 is equal to θ. Thus, the dimensions of the shaped cavity of each end piece 12 and 14 is substantially equal to corresponding dimensions of the spacer 16 when it is inserted between the two end pieces 12 and 14 such that the spacer 16 fits snugly and securely within the cavity and between the two end pieces 12 and 14. Thus, once the spacer 16 is inserted between the end pieces 12 and 14, the spacer 16 is securely positioned within the cavity formed between the end pieces 12 and 14 and cannot be easily removed unless the relative positions of end pieces 12 and 14 are substantially changed.

In accordance with one embodiment of the invention, interchangeable spacers 16 of varying sizes are made available to a physician and the size of the interchangeable spacer 16 may be selected depending on a desired distance between vertebra 18 and 20 (FIG. 1) and/or a desired force each end piece 12 and 14 is to exert against respective adjacent vertebra 18 and 20, as determined by the physician. Thus, if a greater distance between vertebra 18 and 20 for a particular patient is desired, or a greater force is to be exerted against each vertebra 18 and 20 so as to push them apart from each other, the physician may choose a spacer 16 having a body portion with a greater height H₁. In one embodiment, if the dimensions H₃, L₁, L₂ and θ of the cavity of each of the end pieces 12 and 14 remain fixed, then these corresponding dimensions of the spacer 16 should also remain the same so that the spacer 16 still maintains a snug fit within the cavity. Thus, in one embodiment, the dimensions of the spacer 16 corresponding to the values of H₃, L₁, L₂ and θ in FIG. 2B remain fixed, while dimensions corresponding to values H₁, H₂ and L₃ in FIG. 2B are variable in accordance with a desired distance and/or pressure between vertebras 18 and 20.

It is understood that the shape and dimensions of the spacer 16 and the end pieces 12 and 14 illustrated in FIGS. 2A-2C are exemplary only and are not intended to limit the scope of the present invention. Other shapes and sizes of end pieces 12, 14 and spacer 16 are contemplated by the inventors. For example, FIG. 3 illustrates another exemplary spacer 50 having a cylindrical body 52 that narrows to a nose portion 52, with a smaller, cylindrical rear portion 56 extending outwardly from the body portion 52 opposite the nose portion 54. One of ordinary skill in the art would readily understand the complementary shape and dimensions of the internal cavities of corresponding end pieces 12 and 14 so as to function properly with spacer 50, in accordance with another embodiment of the present invention. Variable sizes of spacers 50, each having a different cylindrical diameter, may be selected depending on a desired distance between and/or desired force exerted on vertebras 18 and 20.

FIG. 4 illustrates another exemplary spacer 58 which is spherical or ellipsoidal in shape. The spacer 58 further includes an insertion tool hole 60 configured to function in the same manner as insertion tool hole 36 of spacer 16. It is recognized that a spherical spacer 58 may provide an increased range of motion to corresponding end pieces 12 and 14 with respect to one another, which may be desirable for certain patients under certain circumstances. In this embodiment, the internal cavity of the end pieces 12 and 14 are configured to match the dimensions of the spherical or ellipsoidal spacer 58. As discussed above, the size of the spacer 58 may be selected so as to provide a desired spacing distance D1 in accordance with a desired distance between and/or pressure exerted on vertebras 18 and 20.

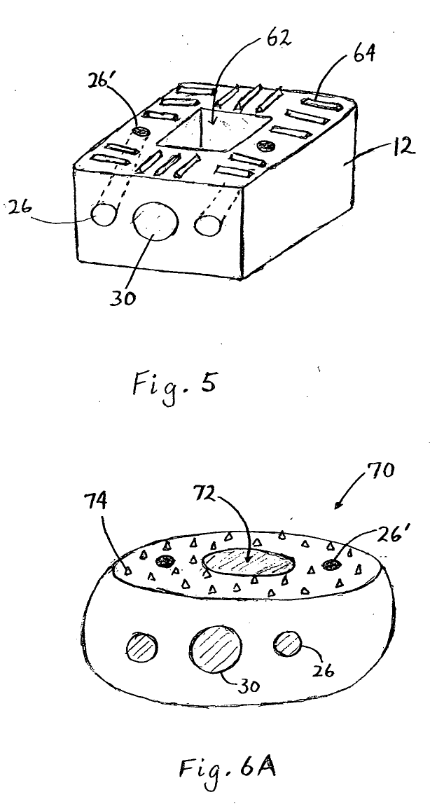

FIG. 5 provides a perspective view of an exemplary end piece 12, in accordance with one embodiment of the invention. The end piece 12 includes the two angled screw holes 26 each having an opening 26′ at a top surface of the end piece 12 and an insertion tool hole 30, as previously described above. In one embodiment, the end piece 12 further includes a window 62 that forms a space or channel through the end piece 12 for allowing insertion of bone graft material and promoting bone growth therethrough. Additional windows, cut-outs or holes (not shown) may also be incorporated into the end piece 12 for allowing and promoting bone growth through the end piece. A plurality of bone anchoring elements 64 is located on the top surface of the end piece 12 for engaging an adjacent vertebra 18 (FIG. 1). An internal cavity or space (not shown) is formed within the end piece 12 having a pre-specified geometric configuration and dimensions so as to geometrically mate with a corresponding spacer (e.g., exemplary spacer 16). The second end piece 14 may be designed and constructed in a similar fashion to the end piece 12, illustrated in FIG. 5.

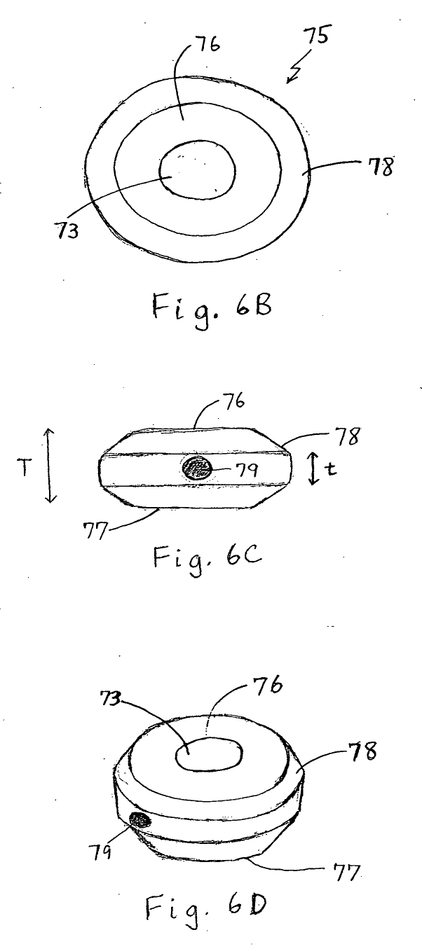

Just as the internal space of the end piece 12 may be varied in accordance with the present invention so too can the external shape and appearance of the end piece 12. FIG. 6A illustrates another exemplary end piece 70 having a donut type shape. The end piece 70 also includes angled screw holes 26 and an insertion tool hole 30, as described above. The end piece 70 further includes a window 72 for allowing bone growth through the end piece 70 and a plurality of anchoring elements 74 for engaging the vertebral bone 18. As can be appreciated, the shape and configuration of the end piece 70 is substantially symmetrical about a central axis and is, therefore, well suited for implantation from any direction of access to the implantation site (e.g., posterior, anterior, lateral, anterolateral, posterolateral).

FIGS. 6B-6C illustrate an exemplary spacer or center piece 75 configured to be inserted between two end pieces that are the same or similar to the donut or ring-shaped end piece 70 of FIG. 6A, in accordance with one embodiment of the invention. The spacer 75 is also substantially disk or donut shaped and therefore substantially symmetrical about a central axis. This shape and design is well suited for implantation from any direction of access to the implantation site (e.g., posterior, anterior, lateral, anterolateral, posterolateral). In other words, because the spacer 75 and the end pieces 70 are substantially symmetrical about a central axis, the stabilization provided by this an expandable implant device should be substantially the same regardless of the direction of insertion and/or final rotational position of the implanted device.

As shown in FIGS. 6B-6D, the spacer 75 includes a center portion having a top surface 76 and a bottom surface 77 and a thickness (T) measured between the top and bottom surfaces. Surrounding the center portion is a tapered portion 78 that tapers gradually to the periphery of the disk shaped spacer 75 to provide a peripheral thickness or height (t). The tapered portion 78 is preferably integral with the center portion. The spacer 75 also includes an optional central window 73 for allowing bone growth therethrough. The spacer 75 further includes an insertion hole 79 configured to receive a prong 101 (FIG. 10) of an insertion tool 100 (FIG. 10) so as to allow manipulation and insertion of the spacer 75 between two end pieces 70 and expand the two end pieces 70 in vivo so as to provide a desired amount of distraction between or within one or more bone and/or tissue structures. The symmetrical shape of the spacer 75 and the end pieces 70 allow for implantation from any one of a plurality of insertion angles without substantially effecting the stabilization provided to the vertebra in any radial direction.

The end pieces 70 each have an internal cavity or contour (not shown) that conforms to the shape and dimensions of the spacer 75 such that the spacer 75 is received and snugly held between the two end pieces 70. The taper peripheral portion 78 of the spacer 75 allows for easy insertion between the end pieces 70 in vivo. In a preferred embodiment of a kit, a plurality of spacers 75 having different thicknesses (T) are made available so that a surgeon can select a spacer having a desired thickness (T) in order to achieve a desired amount of distraction or separation of the end pieces 70.

In one embodiment, the surface contour of the end pieces (e.g., 12, 14, 70) on the surface that meets vertebral bodies 18 and 20 is designed to generally match the contour of the corresponding surfaces of vertebral bodies 18 and 20. As known in the art, the surface contour of the vertebral bodies varies in accordance with differences in contour in different areas of the spine (e.g., cervical, thoracic, lumbar, etc.). Additionally, in one embodiment, the size and shape of the end pieces (e.g., 12, 14, 70) and the spacer 16, 75 may be designed and selected depending on the size of the vertebral bodies 18 and 20 in accordance with the area of the spine being treated (e.g., cervical, thoracic, lumbar, etc.) and/or the size of a particular patient's spine.

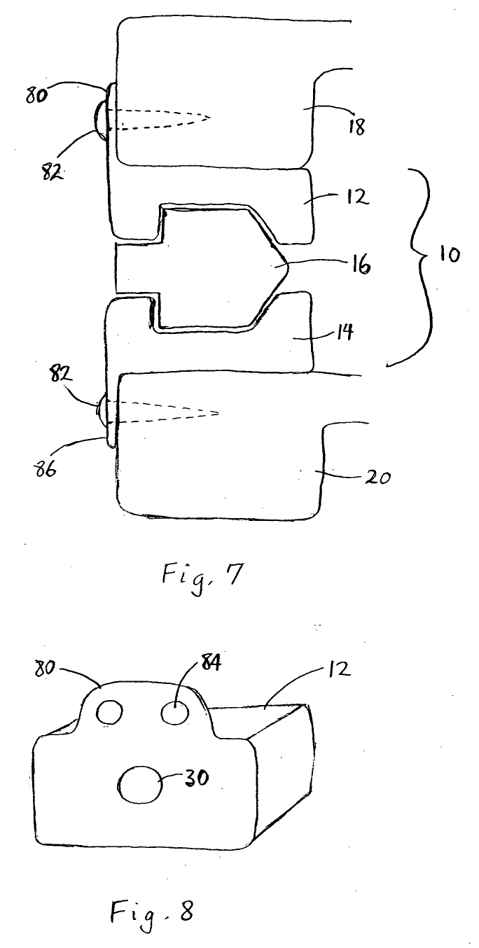

FIG. 7 illustrates a cross sectional view of an expandable implant device 10 similar to that illustrated and described above with respect to FIGS. 1A and 1B. However, instead of the angled screw holes 26 and 28 previously described above, in this embodiment, the end pieces 12 and 14 each include a flange 80 and 86, respectively, for securing the end pieces 12 and 14 to adjacent vertebral bodies 18 and 20, respectively. As shown in FIG. 7, at least one screw 82 passes through the flange 80 and penetrates the vertebra 18, thereby securing the end piece 12 to the vertebra 18. Similarly, at least one screw 82 passes through the flange 86 to secure the second end piece 14 to the vertebra 20.

FIG. 8 illustrates a perspective front view of the end piece 12 of FIG. 7, in accordance with one embodiment of the invention. The end piece 12 includes insertion tool hole 30 and a flange 80 extending upwardly from the front surface of the end piece 12. The flange 80 includes two screw holes 84 through which respective screws 82 may be inserted to secure the end piece to a vertebral body 18. Although not shown in FIG. 8, anchoring elements, such as elements 64 and/or 74 described above, may be placed on the top surface of the end piece 12 and on the rear surface of the flange 80 to engage a vertebral body and prevent the end piece 12 from sliding on the vertebral body.

FIG. 9 illustrates a first insertion tool 90 for inserting end pieces 12 and 14 between two vertebral bodies 18 and 20, after one or more intermediate vertebras (not shown) have been removed. FIG. 9 further illustrates a second insertion tool 100 used for inserted spacer 16 in between the two end pieces 12 and 14 after they have been positioned between the vertebras 18 and 20. The first insertion tool 90 includes first and second prongs 91 and 92 configured to be inserted into respective insertion tool holes 30 and 32 (FIG. 1B). Each of the prongs 91 and 92 include a ball-shaped tip or end 93 and 94, respectively, that function as snap-lock mechanisms when the prongs are fully inserted into the respective holes 30 and 32.

FIG. 10 illustrates a cross-sectional side view of the first end piece 12 wherein the insertion tool hole 30 opens into a channel within the end piece 12. The channel has a complementary shape to that of the first prong 91 and its ball-shaped tip 93 such that when the prong 91 is fully inserted into the channel a narrowing portion 106 in the channel snap-locks the ball-shaped tip 93 within the channel, thereby removably securing the prong 91 to the first end piece 12. The second prong 92 is similarly snap-locked within the second end piece 14.

In another embodiment, the prongs 91 and 92 and corresponding tip 93 and 94 may have a square, rectangular or oval cross-sectional shape. In such embodiments, the corresponding insertion tool holes 30 and 32 of the end pieces 12 and 14, respectively, or at least a portion of the channels accessed by the holes 30 and 32, will have a similar shape (e.g., square, rectangular, or oval).

The first insertion tool 90 includes a fulcrum 96 where the two prongs 91 and 92 meet and which allows the two prongs 91 and 92 to move away and toward one another when two handles 98 located on the opposite side of the fulcrum 96 are moved toward each other or away from each other, respectively. The fulcrum 96 operates under the same principles as the fulcrum in a pair of scissors. However, as shown in FIG. 9, the design and shape of the insertion tool 90 is very different from that of scissors. The first and second prongs 91 and 92 have a bayoneted design wherein each prong is bent at an angle Φ before they meet the fulcrum 96. This bayoneted design allows for easier insertion of the spacer 16 between the end pieces 12 and 14 while the first insertion tool 90 holds and separates the end pieces 12 and 14 in place between the vertebras 18 and 20. Thus, the insertion of the spacer 16 can be performed very quickly and with relatively little resistance as the end pieces 12 and 14 are kept separated by the first and second prongs 91 and 92. After the spacer 16 is inserted into position between the end pieces 12 and 14, the manual separation forces exerted on the end pieces 12 and 14 by the prongs 91 and 92 may be released and the prongs 91 and 92 may be extracted. At this point, the spacer 16 maintains the end pieces 12 and 14 in the desired expanded or distracted state.

A second insertion tool 100 has a single prong 101 configured to be inserted into the insertion hole 36 (FIG. 1B) of the spacer 16. In one embodiment, the prong 101 has a ball-shaped tip 102 that functions to snap-lock the prong 101 in a channel within the spacer 16 when the prong 101 is fully inserted into the hole 36. The channel within the spacer 16 has a complementary shape to that of the prong 101 and its ball-shaped tip 102 such that when the prong 101 is fully inserted into the channel a narrowing portion in the channel snap-locks the ball-shaped tip 102 within the channel, thereby removably securing the prong 101 within the spacer 16. In an alternative embodiment, the snap-lock mechanism may be replaced with threaded means to secure the prong 102 within the spacer 16. In another embodiment, the cross-sectional shape of the prong 101 and corresponding shape of the insertion hole 36 may be square, rectangular or oval, or any other desired shape. The second insertion tool 100 further includes a handle 104 for easy gripping and handling by a physician. As described above, in accordance with one embodiment, the first insertion tool 90 combined with the second insertion tool 100 provide an insertion tool set that can be used to simultaneously insert the first and second end pieces 12 and 14, distract the end pieces to a desired extent, and thereafter insert a selected spacer 16 having a desired dimension between the first and second end pieces 12 and 14 in vivo.

Thus, as illustrated in FIG. 9, after end pieces 12 and 14 are placed onto respective prongs 91 and 92 of the first insertion tool 90, the end pieces 12 and 14 are implanted into the patient and positioned between vertebras 18 and 20 after one or more intermediate vertebras (not shown) and intermediate discs (not shown) have been removed. The end pieces 12 and 14 are then distracted away from one another until they abut and push against respective adjacent vertebras 18 and 20 by squeezing or moving the handles 98 of the insertion tool 90 toward each other. When a desired distance between the end pieces and a desired pressure exerted by the vertebra 18 and 20 pushing back against the end pieces 12 and 14 has been achieved, a physician can determine the size of the spacer necessary to substantially maintain this distance and pressure. After the appropriate size (e.g., H₁) of the spacer 16 has been determined and selected, the selected spacer 16 is inserted into the cavity formed between the end pieces 12 and 14, as described above. Thus, the spacer 16 maintains the end pieces 12 and 14 a desired distance from each other, thereby providing a spinal implant device that is expandable in vivo to a desired height that depends on the size of the spacer 16 selected.

It is appreciated, that the expandable device 10 (expandable cage 10) and the implantation procedure described above is particularly well-suited to be used in minimally invasive surgical procedures. The end pieces 12 and 14 can be inserted through a minimally invasive incision in a “contracted” state (without the spacer 16), expanded in vivo using the insertion tool 90, after which the spacer 16 is inserted to maintain the end pieces 12 and 14 in a desired expanded state.

Although a physician may rely on tactile sensation to feel when a desired amount of pressure or force is being exerted against the end pieces 12 and 14 as they are being separated from one another between the vertebral bodies 18 and 20, in one embodiment, the insertion tool 90 may provide an objective measurement of force and/or distance. FIG. 11A illustrates an end piece insertion tool 90 that further includes a distance and/or force gauge 108 for measuring and providing an indication of the amount of force exerted to separate end pieces 12 and 14 from one another by a desired distance. Thus, as a physician squeezes handles 98 together, prongs 91 and 92 separate from each other, and the gauge 108 indicates a value indicative of the amount of force required to separate the prongs 91 and 92. Alternatively, or additionally, the gauge 108 can indicate a value indicative of the distance of separation between the prongs 91 and 92, or end pieces 12 and 14, when a desired amount of pressure has been achieved. In one embodiment, the gauge 108 is a digital gauge that can provide either or both force and distance readouts. In one embodiment, the digital gauge 108 automatically toggles between distance and pressure values, enabling a physician to obtain objective measurements of both parameters during the surgical implantation procedure. Thus, the physician can rely on these objective measurements to select an appropriate spacer size or use these objective measurements to corroborate his or her tactile, subjective measurements of force and distance.

The gauge 108 further includes a calibration button 110 that can be used to set “zero” reference values when the prongs are positioned as close as possible to each other and/or when no force is exerted to separate them. In one embodiment, the gauge 108 can incorporate torque, pressure or weight sensors (e.g., load cells) (not shown) and circuitry (not shown) coupled to the sensors/load cells to receive a signal from the sensors and provide an output signal to a digital display which indicates a force value. Such sensors, load cells, circuits and displays are known in the art and can be found in commercially available digital weight scales, for example. Additionally, in one embodiment, displacement sensors may also be coupled to the digital circuitry to measure a distance of separation between the prongs 91 and 92 and provide a signal to the digital circuitry. Similar displacement sensors and digital circuitry can be found in digital calipers, for example. The gauge 108 can be easily designed and built by one of ordinary skill in the art, without undue experimentation, so that it performs the desired functions described herein.

FIG. 11B illustrates an end piece insertion tool 150 and corresponding end pieces 160 and 162, in accordance with one embodiment of the invention. The insertion tool 150 is similar to the insertion tool 90 described above, except that the prongs 151 and 152 of the tool 150 have a square cross-sectional shape configured to be inserted into correspondingly shaped holes of end pieces 160 and 162, respectively. As shown in FIG. 11B, the end pieces 160 and 162 each have a cavity 164 configured to receive a correspondingly shaped spacer or center piece (not shown) therein. Each end piece 160 and 162 also includes a window 166 provided to allow bone and/or tissue growth therein and therethrough. The insertion tool 150 also includes a force and/or distance sensor 155. As can be appreciated, the square-shaped prongs 151 and 152 provide additional stability against twisting or rotating of the end pieces 160 and 162 during insertion into a patient and when they are being expanded away from each other in vivo.

FIG. 11C illustrates an end piece insertion tool 170 and corresponding end pieces 180 and 182, in accordance with one embodiment of the invention. The insertion tool 170 is similar to the insertion tool 90 described above, except that the prongs 171 and 172 each terminate with forked prongs 173 and 174, and 175 and 176, respectively. The two end prongs 173 and 174 of the first prong 171 are configured to be received in corresponding holes in the end piece 180. The two end prongs 175 and 176 of the second prong 172 are configured to be received in corresponding holes in the second end piece 182. The end pieces 180 and 182 each have a cavity 184 configured to receive a correspondingly shaped spacer or center piece (not shown) therein. Each end piece 180 and 182 also includes a window 186 provided to allow bone and/or tissue growth therein and therethrough. As can be appreciated, the forked prongs 171 and 172 provide additional stability against twisting or rotating of the end pieces 180 and 182 during insertion into a patient and when they are being expanded away from each other in vivo.

As shown in FIG. 11C, the insertion tool 170 also includes a distance sensor 157. In one embodiment, when a physician determines that an adequate amount of distraction has been achieved based on tactile feel of the amount of force exerted on the handles 98 of the insertion tool 90, the distance sensor will indicate a corresponding distance of separation between the end pieces 180 and 182. This distance reading can be used to select a spacer 16, 75 having a desired size. In a further embodiment, the sensor 158 provides both force and distance readings and, in one embodiment, automatically toggles between distance and force readings. The force readings can be used to corroborate or substitute for the physician's tactile estimation of force. After a desired amount of force is reached during the distraction process, the distance reading is used to select an appropriately sized spacer 16 or 75 to be inserted between the end pieces 180 and 182.

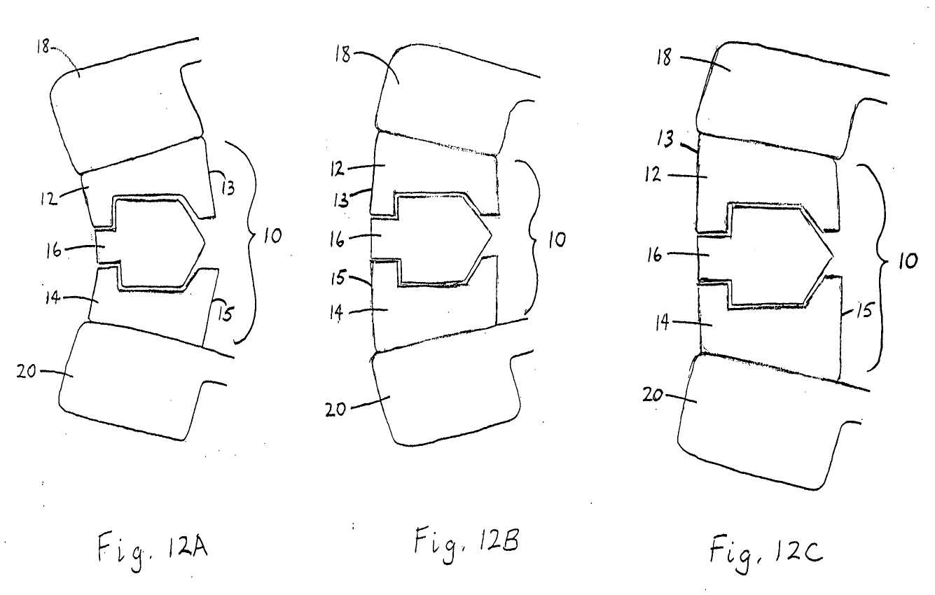

FIGS. 12A-12C illustrate cross-sectional side views of an expandable implant device 10 in accordance with additional embodiments of the invention. In order to emulate a natural or desired curvature of a patient's spine, the first and second end pieces 12 and 14 may be configured such that one end 13, 15 of each end piece 12, 14 is thicker or taller than an opposite end. In FIG. 12A, the end pieces 12 and 14 are configured to emulate an inward curvature (“kyphotic”) of a patient's spine. FIG. 12B illustrates an example of when the end pieces 12 and 14 are configured to emulate a lordotic curvature of the spine. In FIG. 12C, the end pieces 12 and 14 are configured to emulate an asymmetric curvature of the spine.

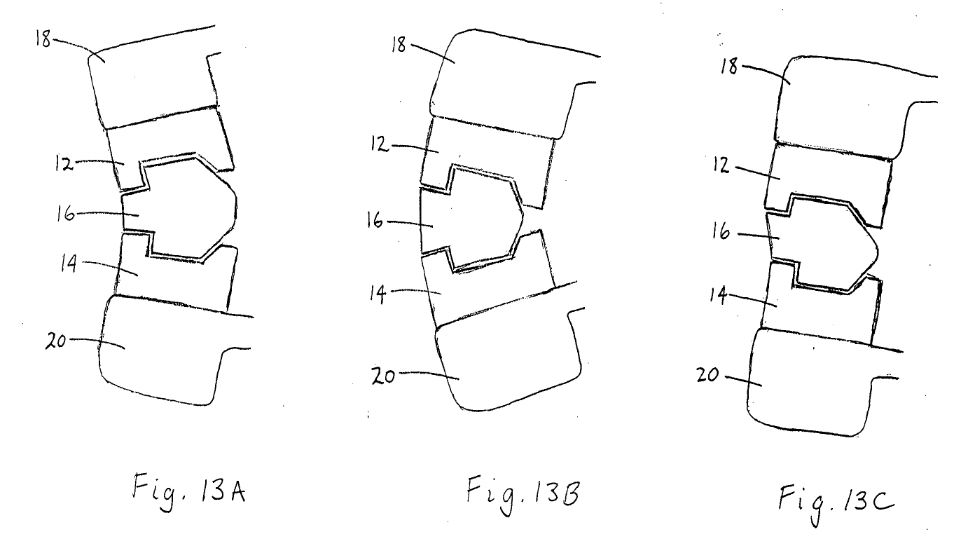

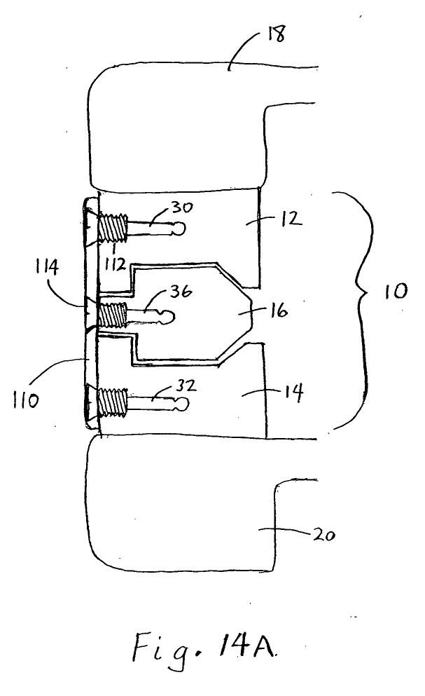

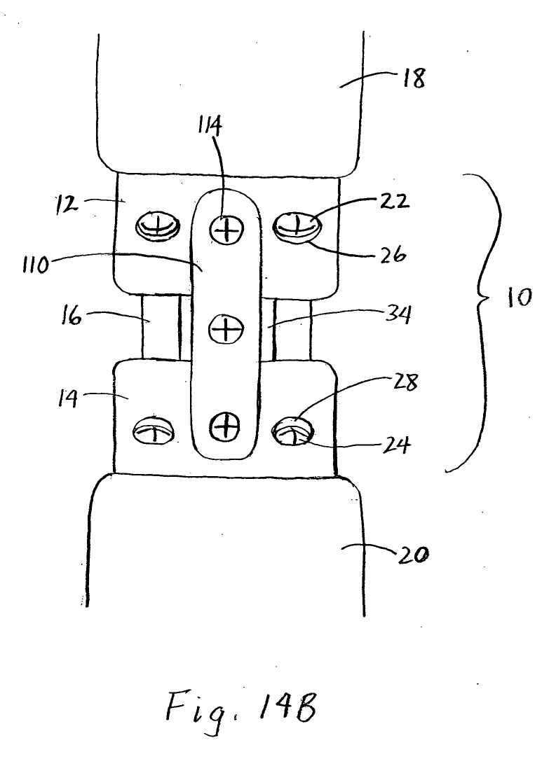

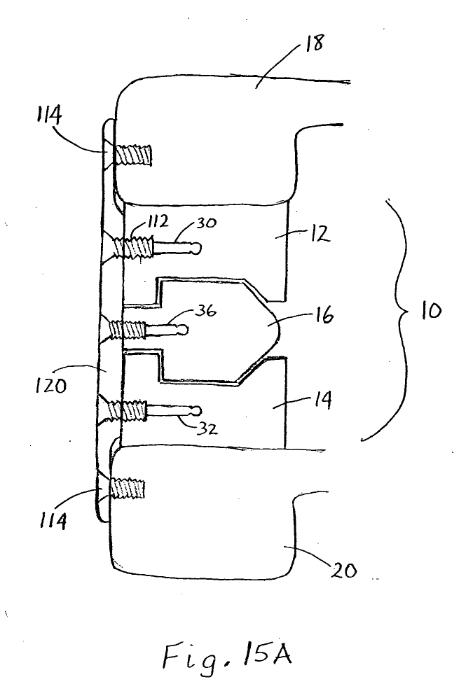

FIGS. 13A-13C illustrate cross-sectional side views of an expandable implant device 10 in accordance with further embodiments of the invention. Instead of altering the geometrical shape of the end pieces 12 and 14, as shown in FIGS. 12A-12C, in these embodiments, the geometric shape of the spacer 16 is changed to emulate the desired spinal curvature. As shown in FIGS. 13A-13C, the exemplary shapes and geometric dimensions of the spacer 16 allow the spacer 16 to snugly and securely fit between the end pieces 12 and 14 while providing a desired curvature. In FIG. 13A, the spacer 16 is configured to emulate an inward curvature (“kyphotic”) of a patient's spine. FIG. 13B illustrates an example of when the spacer 16 is configured to emulate a lordotic curvature of the spine. In FIG. 13C, the spacer 16 is configured to emulate an asymmetric curvature of the spine.

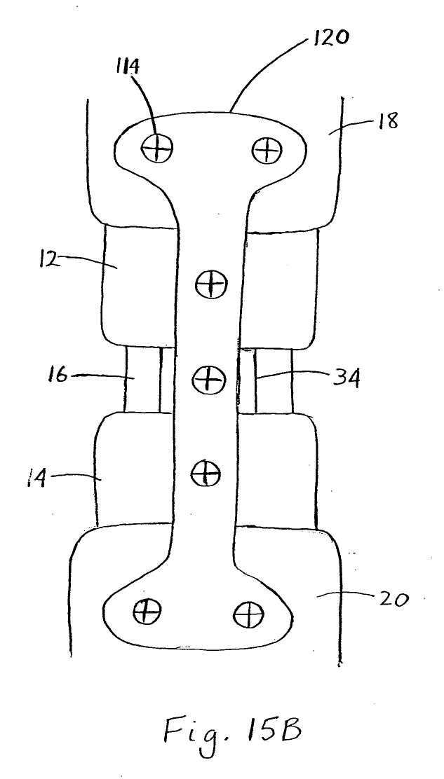

FIG. 14A illustrates a cross-sectional side view of an expandable implant device 10, in accordance with a further embodiment of the invention. This device 10 includes a top plate 110 that may be used to further secure the end pieces 12 and 14 to the spacer 16 so as to prevent or minimize relative movement of these pieces with respect to one another. In one embodiment, biocompatible screws 114 are used to secure the top plate 110 to each of the first and second end pieces 12 and 14, and the spacer 16. The screws 114 are received within internally threaded holes 112 within each of the pieces, as shown in FIG. 14A.

In one embodiment, these internally threaded holes 112 are coaxial with respective insertion tool holes 30, 32 and 36 but have a larger diameter and shorter depth. The larger diameter of the threaded portion of the channels allows insertion tool prongs 91, 92 and 101 to pass unaffected so that they may be fully inserted into their respective pieces 12, 14 and 16, as discussed above. After all the pieces 12, 14 and 16 have been set between vertebral bodies 18 and 20, if the physician desires to further secure the pieces with respect to one another, the top plate 110 may be used for this purpose. Furthermore, the material type, thickness and/or other dimensions of the top plate 110 may be designed and selected to achieve a desired rigidity.

For example, the rigidity of the top plate 110 may be selected to substantially eliminate any movement between the end pieces 12 and 14 and the spacer 16. Or, it may be more flexible to only decrease or limit the amount of movement between these pieces. The top plate 10 may be made from any suitable biocompatible material, or combination of biocompatible materials, to achieve these characteristics. Such biocompatible materials include metals (e.g., titanium), metal alloys, polymers, resins, other known synthetic materials, carbon fibers, etc. Similarly, all of the other pieces and elements of the expandable implant device 10, such as the end pieces 12 and 14, and the spacer 16, may be made from any suitable biocompatible material, or combination of biocompatible materials, known in the art.

FIG. 14B illustrates a perspective front view of the expandable implant device of FIG. 14A, which includes the top plate 110 and screws 114 to secure the top plate 110 to the first and second end pieces 12 and 14, and the spacer 16, thereby securely fastening the end pieces 12 and 14, and the spacer 16 to one another.

FIG. 15A illustrates a cross-sectional side view of another top plate 120 for fastening the expandable implant device 10, in accordance with another embodiment of the invention. The top plate 120 secures the end pieces 12 and 14 and the spacer 16 together in a similar fashion as described above with respect to top plate 110 of FIGS. 14A and 14B. However, top plate 120 is longer than top plate 110 and extends over portions of each of the vertebras 18 and 20. At least one biocompatible screw 114 secures a first end of the top plate 120 to the vertebra 18 and at least one biocompatible screw 114 secures a second end of the top plate 120 to vertebra 20. Additionally, in one embodiment, as illustrated in FIG. 15A, the shape and surface contour of the top plate 120 may be designed to match a desired shape and contour of the corresponding surfaces of the vertebras 18 and 20, end pieces 12 and 14, and the spacer 16. As illustrated in FIGS. 12A-12C, 13A-13C and FIG. 15A, for example, the shapes, sizes and contours of these surfaces may vary in accordance with different spinal curvatures, different vertebra sizes and shapes, different areas of the spine being treated, and/or different embodiments of the expandable implant device 10 of the present invention.

FIG. 15B illustrates a perspective view of the top plate 120 secured to end plates 12 and 14, spacer 16, and vertebras 18 and 20 by a plurality of biocompatible screws 114. It is appreciated that with this configuration, it is not necessary to have angled screws 22 and 24 (FIGS. 1A and 1B) to secure end pieces 12 and 14 to vertebras 18 and 20, respectively. In some embodiments, however, for added fastening strength, it may be desirable to use the angled screws 22 and 24 to secure the end pieces 12 and 14 to their respective vertebras 18 and 20, and, in addition, use the top plate 120 to secure the end pieces 12 and 14, spacer 16 and vertebras 18 and 20 together.

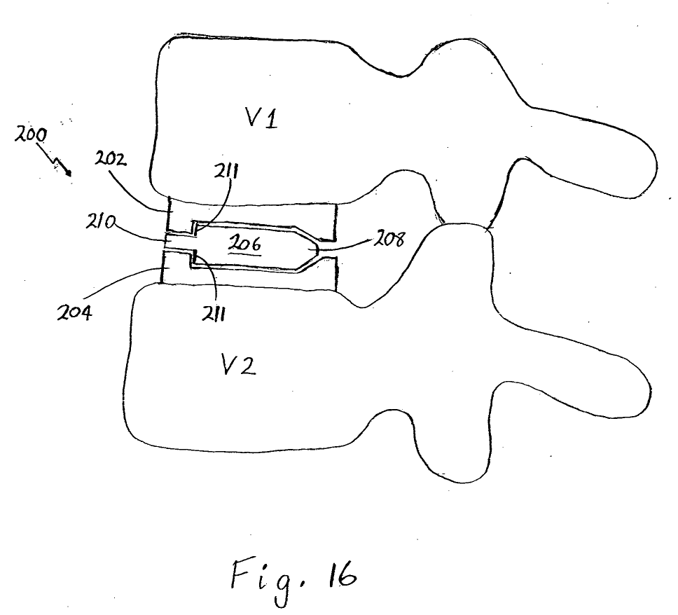

FIG. 16 illustrates a cross-sectional side view of an expandable implant device 200 configured for intervertebral disk replacement. The expandable disk replacement device 200 includes a first end piece 202 configured to be attached and secured to a first vertebral body V1 and a second end piece 204 configured to be attached and secured to a second vertebral body V2. After the damaged disk has been removed using known techniques, the end pieces 202 and 204 are inserted in a contracted state (without a spacer 206) between the first and second vertebral bodies V1 and V2. The spacer 206 is configured to be inserted between the first and second pieces 202 and 204, respectively. Each of the first and second end pieces 202 and 204, respectively, and the interchangeable spacer 206 can be surgically implanted in the same manner using the same or similar tools and techniques described above. By selecting a spacer 206 having desired dimensions, the overall height of the implant device 200 can be custom tailored to fit the needs of a particular patient and/or achieve a desired amount of distraction between the first and second vertebral bodies V1 and V2. As shown in FIG. 16, the spacer 206 has a tapered leading edge 208 for easy insertion into the space between the first and second end pieces 202 and 204. The rear of the spacer includes a narrow portion 210 which forms two backstop walls 211 that step up to a wider main body of the spacer 206. The backstop walls 211 interlock with corresponding walls within the internal cavity formed between the first and second pieces 202 and 204, respectively, to prevent the spacer 206 from backing out of the cavity. The first and second pieces 202 and 204, and the spacer 206 may be made from any desired biocompatible material and can be secured and placed into their respective positions using any of the techniques described above. In one embodiment, the first and second pieces 202 and 204, and the spacer 206, have insertion holes (not shown) for coupling to respective insertion tools for facilitating implantation as described above with respect to FIGS. 9-11C. It is appreciated that the expandable device 200 can be modified for use as an intra-disk augmentation device that replaces only a portion (e.g., nucleus) of a damaged disk.

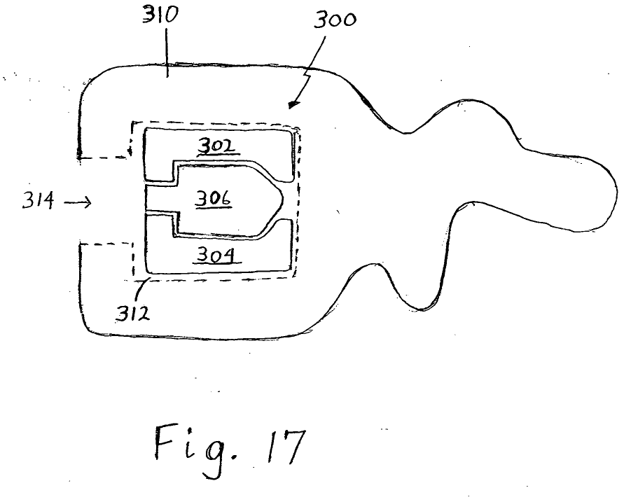

FIG. 17 illustrates a cross-sectional side view of an expandable implant device 300 configured for intravertebral augmentation. The expandable device 300 includes two end pieces 302 and 304 that are simultaneous inserted into a vertebral body 310 through an insertion site or hole 314 prepared on the vertebral body 310 prior to implantation of the end pieces 302 and 304 therein. It is understood that the hole 314 may be prepared in other locations of the vertebral body accessed from any desired direction. The end pieces 302 and 304 are inserted in a contracted state (without a spacer) with minimum separation from each other so that they fit through the insertion site 314. After they are positioned within the vertebral body 310, the end pieces 302 and 304 are distracted away from each other using the insertion tool 90 (FIG. 9), for example, and a desired level of distraction is determined by a physician, as described above. A spacer 306 having an appropriate size is then selected and inserted between the end pieces 302 and 304, in a similar manner to that described above, thereby separating and maintaining the end pieces 302 and 304 in a desired expanded state in vivo.

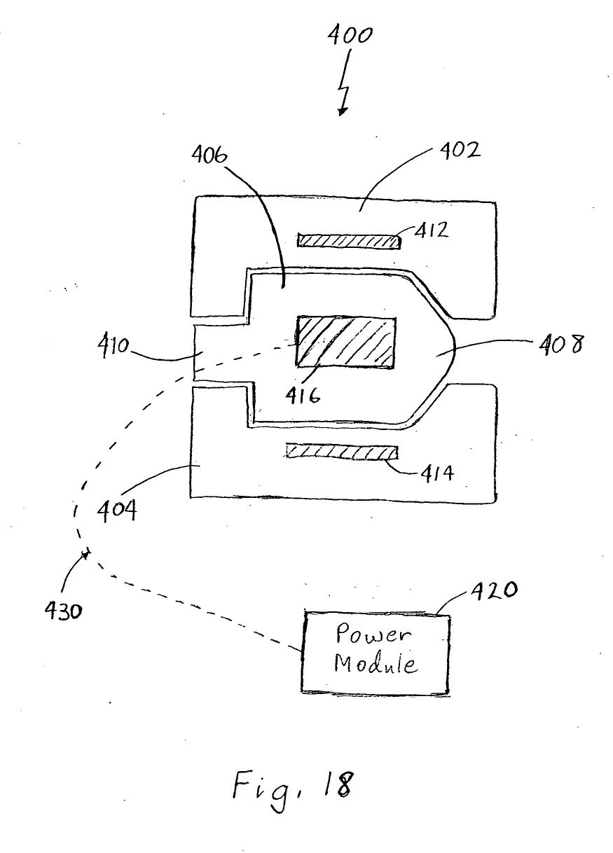

FIG. 18 shows a cross-sectional side view of an expandable implant 400 having magnetic elements 412, 416 therein, in accordance with one embodiment of the invention. It is understood that the figures herein are not necessarily drawn to scale. The expandable implant 400 can be configured for use in any of the applications described above (e.g., vertebral body replacement, intervertebral disk replacement, intravertebral body augmentation). As shown in FIG. 18, the first piece 402 includes a first optional magnetic element 412 embedded or encapsulated therein and the second piece 404 includes a second optional magnetic element 414 embedded or encapsulated therein. The interchangeable spacer 406 includes a magnetic element 416 configured to induce a magnetic force between itself and the first and second magnetic elements 412 and 414. The polarity of the magnets 412, 414 and 416 can be selected such that the magnetic forces induced between magnets 416 and 412 and between magnets 416 and 414 can both be repelling forces, one repelling force and one attraction force, or both attraction forces. If both are repelling forces, this configuration provides a maximum level of repulsion (or “shock absorption”) against axial compression forces. If one force is attraction and the other is repulsion, this configuration provides an intermediate level of repulsion while magnetically holding the spacer 406 against either the first end piece 402 or second end piece 404. If both provide attraction forces, the interchangeable spacer 406 is magnetically attached and held to each of the first and second pieces 402 and 404 to provide maximum holding strength between the spacer 406 and each of the first and second end pieces 402 and 404.

In one embodiment, the magnetic elements 412 and 414 may simply be a suitable metal that is affected by magnetic forces generated by the center magnet 416. Alternatively, the end pieces 402 and 404 may themselves be made from such a suitable metal or metal composition material, eliminating the need for the magnetic elements 412 and 414 embedded or encapsulated therein. Additionally, in other embodiments, the magnets 412 and/or 416 can each comprise two or more magnetic elements to provide desired attraction, repulsion characteristics and combinations thereof. For example, in cases where asymmetrical forces are desired to compensate for asymmetrical misalignment of the spine, magnetic elements can be strategically placed to provide the desired compensation forces.

The amount of repulsion or attraction forces provided by the magnets 412, 414 and/or 416 can be tailored for each application and the particular needs of each patient. Various types, sizes, shapes of permanent magnets and magnetic alloys exhibit different magnetic attraction and repulsion forces. Additionally, the magnets 412, 414 and 416 can be encased or embedded in various biocompatible materials (e.g., titanium, titanium alloys, ceramics, polymers, etc.) to provide various levels of magnetic shielding to further control the magnetic forces exerted between the magnets. Alternatively, one or more of the magnets 412, 414 and/or 416 can comprise electromagnets to control the amount of distraction force between the magnets by controlling the electric current applied to the electromagnets. In one embodiment, the center piece magnet 416 comprises an electromagnet. As shown in FIG. 18, in this embodiment, the electromagnet 416 is coupled to a power module 420 via a two-wire connection cable 430.

The power module 420 may be designed to be fully implanted subcutaneously at an advantageous location within a patient or configured to be attached to the skin of the patient with the cable 430 extending transcutaneously through the patient's skin and providing electrical contact with the fully implanted electromagnet 416 and an external power module 420. In one embodiment, the power module 420 is configured to be fully implanted subcutaneously and communicates with external devices via radio frequency (RF) telemetry. Various types of implantable RF telemetry devices are known in the art, for example, to monitor a patient's heart rate or condition, glucose monitoring, etc. Similar types of RF telemetry systems can be utilized in accordance with the present invention to control the power supplied to the electromagnet 416.

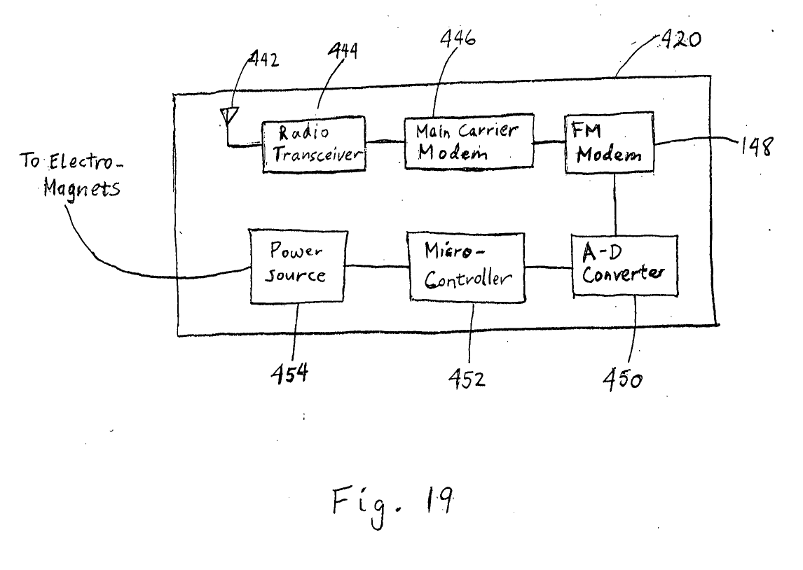

In one preferred embodiment, the power module 420 includes a power source (e.g., a battery) and additional circuitry for controlling the amount, duration and/or intervals in which current is supplied to the electromagnet 416. As shown in FIG. 19, in one embodiment, the power module 420 includes a radio frequency antenna 442, a radio transceiver 444, a main carrier modem (modulation/demodulation device) 446, a FM modem 448, an analog-to-digital (A-D) converter 450, and a microcontroller 452 coupled to a power source 454. The antenna 442 and transceiver 444 enable communications via radio telemetry with an external device that can send command signals to control the power, duration and frequency of power supplied to the electromagnet 416. Incoming radio frequency (RF) command and/or data signals are demodulated by the modems 446 and 448 and supplied to the A-D converter 450, which converts analog waveforms into digital signals. The digital signals are then provided to microcontroller 452 for processing. The microcontroller 452 is coupled to the power source 454 for controlling the amount, duration, frequency, on/off times, etc. of the power source 454. The microcontroller 452 can further include a programmable read only memory (PROM) (not shown), a random access memory (RAM) (not shown) and a microprocessor (not shown) for executing program instructions stored in the PROM, processing received data, storing any desired data, controlling the operation of the power source 454, and/or communicating with external devices (e.g. a monitor and/or controller with keypad). Each of the electronic components or modules 442-454 may be separate components or, alternatively, integrated into one or more integrated circuit (IC) chips.

In one embodiment, the power module 420 may be similar to the implantable power module (IPM) disclosed in U.S. Pat. No. 6,894,456, the entirety of which is incorporated by reference herein. Thus, the power module 420 can be configured to be implanted in a human for extended periods of time, and, contained within a hermetic biocompatible case having a highly reliable external connector (e.g., an external hermetic plug), a source of electrical power, a control circuit, an inductive recharging coil (in the case of secondary batteries and/or capacitors), a homing device for precisely locating the implanted module, and safety devices and design aspects to protect the patient in which the IPM is implanted. The source of power or “battery” may be one or more primary or secondary cells, a capacitor, a nuclear-heated thermopile or nuclear battery, or combinations of the above. Lithium cells are generally considered the best overall compromise of energy capacity, reliability, safety, and rate capability. However, hybrid devices having properties of both lithium cells and super capacitors may have improved performance depending on the demands of the device it is powering. While the term “battery” is used for convenience herein, its meaning may include any electrical storage or generation device.

It is further understood that the power module 420 need not continuously supply current to the electromagnet 416. Power may be supplied intermittently or only at desired times for desired durations while the power module 420 is sufficiently charged to provide a desired amount of current. In one embodiment, the microcontroller 452 may be programmed to monitor the amount of power available in battery 454 and communicate via RF telemetry to an external device when the battery 454 needs to be recharged (e.g., via magnetic induction). Such an external device (not shown) may be configured or integrated into a wrist-watch type of device, for example, so as to provide a convenient monitor to a patient without disrupting normal everyday activities. Other types of external devices (e.g., cell phones, personal digital assistants (PDA's), etc.) also may be advantageously utilized to communicate with and provide control signals to the power module 420. As battery technologies continue to improve, the power source 454 will be able to continuously provide power to the electromagnet 416 for longer periods of time before recharging is required. For example, the power source 454 may utilize thermoelectric nanomaterials that are adapted to store heat, e.g., from the patient's body, and convert that heat into electricity to be used for recharging the power source 420.

In a further embodiment, the microcontroller 452 may be programmed to monitor axial loads exerted onto the implant 400 (FIG. 18) via, for example, pressure sensors (not shown), and/or changes in magnetic flux between the electromagnet 416 and magnetic elements 412 and 414, for example, or other magnets (not shown), located on the first and second pieces 402 and 404. As axial loads increase, the first and second end pieces 402 and 404 will be pushed closer together and the space between them will decrease. By measuring the change in distance between the first and second pieces 402 and 404, for example, the microcontroller 452 can monitor the axial loads exerted onto the implant 400. One exemplary method of measuring the spacing between the first and second end pieces 402 and 404 is disclosed in U.S. publication no. 2005/0010301 A1, the entirety of which is incorporated by reference herein. By monitoring the axial loads exerted onto the device, the microcontroller 452 may turn on and/or control the level of current supplied to the electromagnet 416 and thereby provide a suitable distraction or repelling force between the spacer 406 and the end pieces 402 and 404 to compensate for the increased axial loads. For example, when a patient is undergoing strenuous physical activity (e.g., running), the microcontroller 452 may sense increased axial loads on the spine and thereafter turn on the electromagnet 416 or increase the current level supplied in order to provide increased distraction or cushioning forces to compensate for the axial loads changes. As another example, a patient may desire magnetic distraction forces to be applied to the patient's spine for only a few minutes or hours a day, in order to stretch and/or provide relief from compression and axial loads applied to the spine throughout the day.

Additionally, magnetic forces may provide therapeutic value to help relieve muscle strain and/or heal damaged or tired muscles. The therapeutic properties of magnetic fields are known in the art and described, for example, in U.S. Pat. No. 5,707,333.

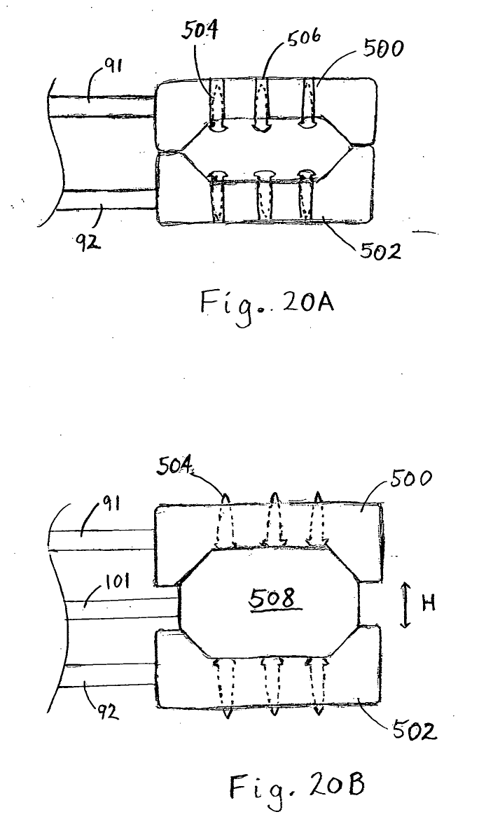

FIG. 20A illustrates a cross-sectional side view of two end pieces 500 and 502 having retracted pins 504 located in pin sockets 506 on the end pieces 500 and 502. When the two end pieces 500 are inserted into a patient via a minimally invasive procedure, for example, the pins are in a retracted state as shown in FIG. 20A. In this retracted state, the pins do not hinder, grab or scrape against bone or tissue during insertion and positioning of the end pieces 500 and 502 between two vertebral bodies. After the end pieces 500 and 502 are positioned as desired, a spacer 508 is inserted between the end pieces 500 and 502, thereby expanding the end pieces 500 and 502 in vivo. As the spacer 508 is inserted between the end pieces 500 and 502, the spacer 508 pushes the pins 504 outwardly such that they protrude out of respective surfaces of the end pieces 500 and 502, so as to provide anchoring elements 504 that engage respective bone surfaces (not shown). Thus retractable anchoring elements 504 do not hinder the insertion and positioning of end pieces 500 and 502 prior to expansion, but provide a bone anchoring function after expansion of the end pieces 500 and 502 by a spacer 508 in vivo.

Various preferred embodiments of the invention have been described above. However, it is understood that these various embodiments are exemplary only and should not limit the scope of the invention. Various modifications of the preferred embodiments described above can be implemented by those of ordinary skill in the art, without undue experimentation. These various modifications are contemplated to be within the spirit and scope of the invention.

What is claimed is:

1. A method of inserting an implantable device having two end pieces and a spacer configured to be positioned between the two end pieces, the method comprising:
simultaneously inserting the two end pieces into a surgical site of a patient;
applying a force to separate the end pieces away from each other in vivo;
inserting the spacer between the two end pieces while maintaining the separation force between the two end pieces; and
releasing the separation force, wherein the spacer maintains a desired separation distance between the two end pieces.

2. The method of claim 1 further comprising:
determining when a desired amount of force has been exerted to separate the end pieces; and
determining a distance of separation between the end pieces when the desired amount of force is exerted, wherein the spacer is selected from a plurality of spacers having different sizes based on the distance of separation.

3. The method of claim 2 wherein the force is determined based on a tactile feel of a surgeon while manually applying the force via an insertion tool used to insert the two end pieces.

4. The method of claim 2 wherein the force is determined using a force gauge coupled to an insertion tool used to insert the two end pieces.

5. The method of claim 2 wherein the distance is determined using a distance gauge coupled to an insertion tool used to insert the two end pieces.

6. The method of claim 1 wherein the surgical site is a site located between two vertebral bodies after at least one intermediate vertebral body has been removed, the method further comprising securing the two end pieces to respective ones of the two vertebral bodies.

7. The method of claim 1 wherein the surgical site is a site located between two vertebral bodies after an intermediate disk has been removed, the method further comprising securing the two end pieces to respective ones of the two vertebral bodies.

8. An implantable device that is expandable in vivo, comprising:
a first end piece having a top surface and a contoured bottom surface;
a second end piece having a bottom surface and a contoured top surface, wherein the first and second end pieces are configured to be inserted simultaneously into a surgical site of a patient with the contoured bottom surface of the first end piece facing the contoured top surface of the second end piece; and
a spacer configured to be inserted between the first and second end pieces in vivo, the spacer having a first contoured surface configured to engage the contoured bottom surface of the first end piece, and a second contoured surface configured to engage the contoured top surface of the second end piece.

9. The implantable device of claim 8 wherein:
the first end piece comprises at least one first hole configured to receive at least one first prong of a first insertion tool; and
the second end piece comprises at least one second hole configured to receive at least one second prong of the first insertion tool, wherein the first and second end pieces are configured to be inserted simultaneously into a surgical site within a patient and thereafter separated from each other in vivo by the first and second prongs.

10. The implantable device of claim 9 wherein the spacer comprises:
a third hole configured to receive a prong of a second insertion tool; and
a tapered leading edge portion to facilitate insertion of the spacer between the first and second end pieces in vivo.

11. The implantable device of claim 8 wherein:
the first end piece comprises a first window formed through the top surface and contoured bottom surface of the first end piece; and
the second end piece comprises a second window formed through the bottom surface and contoured top surface of the second end piece; and
the spacer comprises a third window formed through the first and second contoured surfaces of the spacer, wherein when the spacer is inserted between the first and second end pieces in vivo, the first, second and third windows form one continuous window through the implantable device so as to allow bone or tissue growth therethrough.

12. The implantable device of claim 8 wherein:
the first end piece comprises a first plurality of retractable anchoring elements; and
the second end piece comprises a second plurality of retractable anchoring elements, wherein the first and second plurality of retractable anchoring elements are maintained in a retracted state during insertion of the first and second end pieces into the surgical site, and retracted outwardly through the top and bottom surfaces, respectively, when the spacer is inserted between the first and second end pieces in vivo.

13. The implantable device of claim 8 wherein the spacer and the first and second end pieces have shapes which are substantially symmetrical about a central axis.

14. The implantable device of claim 8 further comprising at least one magnetic element for providing a magnetic force between the spacer and at least one of the first and second end pieces.

15. The implantable device of claim 14 wherein the magnetic force comprises a magnetic repulsion force for dynamically cushioning axial loads between the spacer and at least one of the first and second end pieces.

16. The implantable device of claim 14 wherein the magnetic force comprises a magnetic attraction force between the spacer and at least one of the first and second end pieces.

17. The implantable device of claim 8 wherein the spacer and the first and second end pieces are configured to fit together so as to provide a desired curvature.

18. A method of providing an expandable spinal implant device that is customizable in real-time during surgery, comprising:
providing a first end piece having a top surface and a contoured bottom surface;
providing a second end piece having a bottom surface and a contoured top surface, wherein the first and second end pieces are configured to be inserted simultaneously into a surgical site of a patient with the contoured bottom surface of the first end piece facing the contoured top surface of the second end piece;
providing a plurality of interchangeable spacers of various heights, wherein each spacer is configured to be inserted between the first and second end pieces in vivo after the first and second end pieces have been inserted, each spacer having a first surface, configured to engage the contoured bottom surface of the first end piece, and a second surface configured to engage the contoured top surface of the second end piece, wherein each one of the plurality of spacers is configured to maintain a specified amount of separation between the first and second end pieces.

19. The method of claim 18 further comprising providing an insertion tool set configured to simultaneously insert the first and second end pieces into the surgical site, the insertion tool set being further configured to distract the end pieces in vivo to a desired extent and thereafter insert a selected one of the plurality of spacers between the first and second end pieces in vivo.

20. The method of claim 19 further comprising providing a distance gauge coupled to the insertion tool set, wherein the distance gauge indicates a distance of separation between the first and second end pieces when a desired force has been exerted to distract the first and second end pieces in vivo.
